(19) United States
(12) Patent Application Publication
(10) Pub. No.: US 2017/0029757 A1
(43) Pub. Date: Feb. 2, 2017

(54) CULTURE VENT PLUG, LID BODY KIT, AND FRAME BODY FOR CULTURE VENT PLUG

(71) Applicant: TAIYO KOGYO CO., LTD., Tokyo (JP)

(72) Inventors: Yosuke KAWAUCHI, Tokyo (JP); Hiromi AKATSUKA, Tokyo (JP)

(73) Assignee: TAIYO KOGYO CO., LTD., Tokyo (JP)

(21) Appl. No.: 15/302,285

(22) PCT Filed: May 12, 2015
(86) PCT No.: PCT/JP2015/063579

§ 371 Date: Oct. 6, 2016

(30) Foreign Application Priority Data
May 16, 2014 (JP) 2014-102574

(51) Int. Cl. C12M 1/00

(52) U.S. Cl. CPC C12M 23/38

(57) ABSTRACT

A culture vent plug, a lid body kit, and a frame body for the culture vent plug are provided having a configuration which prevents air permeability from lowering when a vessel opening portion is blocked and excellent in cleanability and sterilizability. A vent plug body capable of securing ventilation between inside and outside a culture vessel; and a frame body including a holding portion holding the vent plug body, and an attachment portion capable of being attached to and detached from a vessel opening of the culture vessel are included, and the vent plug body is held by the holding portion so as to be capable of being repeatedly attached to and detached from the frame body.

### TECHNICAL FIELD

The present invention relates to a culture vent plug, a lid body kit, and a frame body for a culture vent plug.

### BACKGROUND ART

Conventionally, a vent plug including a porous vent plug body has been used as a plug of a culture vessel for culturing a microorganism or the like. The porous vent plug body includes a vent hole capable of achieving air permeability satisfying the aerobic property of a microorganism or the like to be cultured.

For example, Patent Literature 1 discloses an invention of a plug body of a culture and bleeding vessel constituted of a foamed elastic body where an independent pore structure in which pores are isolated from each other by a barrier and a continuous pore structure in which a portion of the barrier isolating pores from each other is destroyed are mixedly exist. Specifically, a vent plug configured by using an elastic foamed molding such as silicone rubber is disclosed.

For details, FIG. 1 of Patent Literature 1 discloses a vent plug (hereinafter also referred to as conventional technique 1) which is an integrated molding made of one material, configured by using an elastic foamed molding such as silicone rubber.

Conventional technique 1 is an integrated molding made of one material, configured by using an elastic foamed molding. Therefore, when conventional technique 1 is press-fitted to the inner peripheral surface of a vessel opening portion of a culture vessel and blocks the vessel opening portion, conventional technique 1 is narrowed in the vicinity of the vessel opening portion. Thus, there is a problem that a vent hole provided inside the elastic foamed molding is crushed and air permeability is lowered.

FIG. 2a of Patent Literature 1 discloses a plug type vent plug (hereinafter also referred to as conventional technique 2) configured by fitting a disc-shaped vent plug body (plug body) to a frame body (frame member) by adhesion or integral molding. Conventional technique 2 forms a structure where the vent plug body (plug body) is integrally fixed to the inner peripheral surface of the frame body (frame member), which is a cylindrical body.

The frame body in conventional technique 2 is constituted of approximately spindle-shaped plastic relatively rich in elasticity such as polyethylene or hard rubber (see paragraph in Patent Literature 1). Therefore, when the vent plug, conventional technique 2, is press-fitted to the inner peripheral surface of a vessel opening portion of a culture vessel and blocks the vessel opening portion, the vent plug body can be protected by the frame body. Therefore, in the above blocked state, the vent plug body in conventional technique 2 is less likely to be narrowed than that in conventional technique 1, and the problem that the vent hole is crushed can be solved.

### CITATION LIST

### Patent Literature

### Patent Literature 1: JP1996-140660 A

### SUMMARY OF INVENTION

### Technical Problem

However, conventional technique 2 has the following problem. That is, since conventional technique 2 is configured of the frame body and the vent plug body, the structure of conventional technique 2 is more complex than the structure of a vent plug like conventional technique 1, which is integrally made of one material. Therefore, conventional technique 2 has the problem that it is difficult to be cleaned.

Usually, a microorganism or the like is aseptically cultured so that bacteria, dust, and the like are not mixed with the microorganism or the like. Therefore, in the case where the vent plug is repeatedly used, it is required that the vent plug be sufficiently cleaned. Therefore, deterioration in cleanability is a problem from the viewpoint of performing aseptic culture.

In addition, in the case of repeatedly using the vent plug, the vent plug is usually subjected to a high-temperature sterilization process after being cleaned. Since moisture adheres to the vent plug when the vent plug is cooled to room temperature after being subjected to the high-temperature sterilization process, it is necessary to sufficiently dry the vent plug. However, since the structure of the vent plug in conventional technique 2 is more complicated than that in conventional technique 1, moisture is more likely to remain in a small part of the vent plug and the vent plug may not be sufficiently dried in some cases. When the vent plug to which moisture adheres is used for a culture vessel, contamination, that is, mixture of bacteria into culture, is likely to occur, which is a problem.

The present invention is made in view of the above problems. That is, the present invention provides a culture vent plug, a lid body kit, and a frame body for a culture vent plug having a configuration which prevents air permeability from lowering when a vessel opening portion is blocked and excellent in cleanability and sterilizability.

### Solution to Problem

A culture vent plug of the present invention includes: a vent plug body capable of securing ventilation between inside and outside a culture vessel; and a frame body including a holding portion holding the vent plug body, and an attachment portion capable of being attached to and detached from a vessel opening of the culture vessel, wherein the vent plug body is held by the holding portion so as to be capable of being repeatedly attached to and detached from the frame body.

A lid body kit of the present invention includes: a plurality of lid bodies each of which is used for the culture vent plug of the present invention and is repeatedly and detachably attached to an exposed end portion of the frame body exposed from the vessel opening, the lid bodies differing from each other in color of an outer peripheral surface.

A frame body for a culture vent plug of the present invention used for the culture vent plug of the present invention includes a holding portion repeatedly and detachably holding the vent plug body; and an attachment portion repeatedly and detachably attached to a vessel opening of a culture vessel.

### Advantageous Effects of Invention

In the culture vent plug according to the present invention, since the vent plug body is attached to the frame body, the vent plug body is less likely to be narrowed when the vessel opening portion is blocked.

In addition, since the vent plug body is held so as to be capable of being repeatedly attached to and detached from the frame body, it is possible to separate the vent plug body from the frame body after completion of culture and to separately clean the frame body and the vent plug body. Therefore, a person who cleans the culture vent plug can easily and sufficiently clean even a small part of the culture vent plug.

In addition, in the culture vent plug according to the present invention, it is possible to separately subject the frame body and the vent plug body to a high-temperature sterilization process and to dry them separately, and moisture adhering to the culture vent plug after the high-temperature sterilization process is favorably removed due to the simplified structure.

The culture vent plug according to the present invention becomes a clean culture vent plug by fitting together again the frame body and the vent plug body which have been sufficiently cleaned and sterilized, and the culture vent plug can be repeatedly used.

The lid body kit according to the present invention includes a plurality of lid bodies differing from each other in color of the outer peripheral surface, and a plurality of culture vessels to which culture vent plugs in which the lid bodies are used are attached can be distinguished from each other by the color of the lid body. For example, by changing the color of the lid body for each culture condition with respect to a plurality of culture vessels under different culture conditions, the culture vessels different from each other in culture condition can be easily and visually distinguished from each other.

The frame body for a culture vent plug according to the present invention can be handled separately from the vent plug body. Therefore, it is possible to meet the demand of cleaning, sterilizing at high temperature, drying, or replacing only the frame body in the culture vent plug according to the present invention.

### BRIEF DESCRIPTION OF DRAWINGS

The above-described objects, and other objects, features, and advantages will be more apparent from preferred embodiments which will be described below, and the following accompanying drawings.

FIG. 1 is a view of the entirety of a culture vent plug exemplifying a culture vent plug according to a first embodiment of the present invention, and illustrates a state of being attached to a culture vessel.

FIG. 2 is a side view of the culture vent plug according to the first embodiment of the present invention, and illustrates a state where a vent plug body and a lid body are detached from a frame body.

FIG. 3 is a cross-sectional view taken along line III-III of the culture vent plug according to the first embodiment illustrated in FIG. 1.

FIG. 4 is a cross-sectional view of a culture vent plug exemplifying a culture vent plug of a second embodiment of the present invention.

FIG. 5 is a side view of a culture vent plug according to a third embodiment of the present invention, and illustrates a state where a vent plug body and a lid body are detached from a frame body.

FIG. 6 is a cross-sectional view of a culture vent plug exemplifying a culture vent plug according to a fourth embodiment of the present invention.

### DESCRIPTION OF EMBODIMENTS

Hereinafter, the present invention will be described with reference to the drawings. In all the drawings, similar constituents are denoted by identical reference signs and overlapping descriptions will be appropriately omitted.

Note that in the description of the present invention, a description will be given defining the up-down direction as illustrated in some cases. However, the up-down direction is defined for descriptive purposes in order to easily explain the relative relation of constituents, and not for limiting the direction when a product in which the present invention is implemented is manufactured or used.

The following and the like are permitted: various constituents of the present invention are not necessarily independent from each other; a plurality of constituents is formed as one member; one constituent is formed of a plurality of members; a certain constituent is part of another constituent; and a certain constituent and another constituent have a part in common.

In the description of the present invention, the end on the culture vessel side of the culture vent plug of the present invention in the state of being attached to the culture vessel is appropriately referred to as a front end, and the end on the side opposite to the front end is appropriately referred to as a base end. In addition, a predetermined length area including the above base end is referred to as a base end portion, and a predetermined length area including the above front end is referred to as a front end portion. In addition, the direction connecting the above front end and the above base end may be referred to as the front-end to base-end direction in some cases.

In addition, in the description of the present invention, the term, inner peripheral surface or outer peripheral surface may be appropriately used in some cases. Unless otherwise explained, the above term, inner peripheral surface, means a surface facing the axial center of the frame body, and the above term, outer peripheral surface, means a face not facing the axial center. In addition, the up-down direction with respect to the lid body is not defined by the gravity direction but indicates the relative positional relation in the lid body. More specifically, in the lid body attached to the frame body, the exposed end side of the frame body is referred to as up and the inserted end side is referred to as down.

### First Embodiment

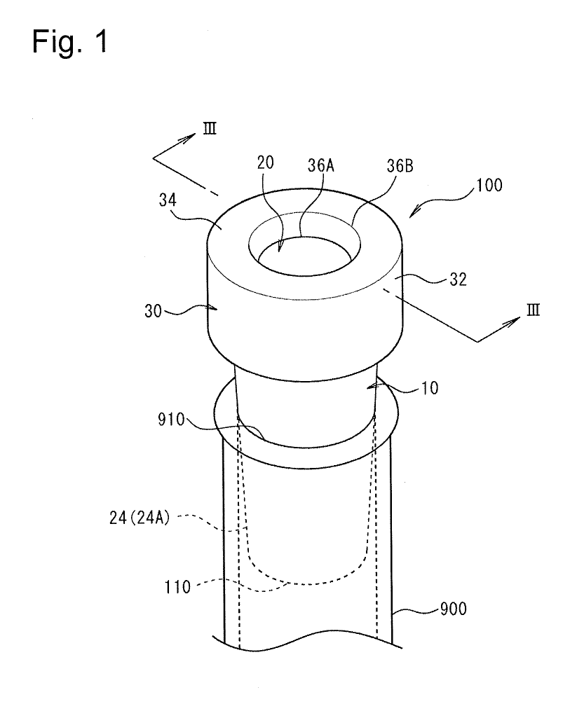

Hereinafter, a culture vent plug 100, which is a first embodiment of the culture vent plug of the present invention, will be described with reference to FIGS. 1 to 3.

FIG. 1 is a view of the entirety of the culture vent plug 100 exemplifying the culture vent plug according to the first embodiment of the present invention, and illustrates a state where the culture vent plug 100 is attached to a culture vessel 900.

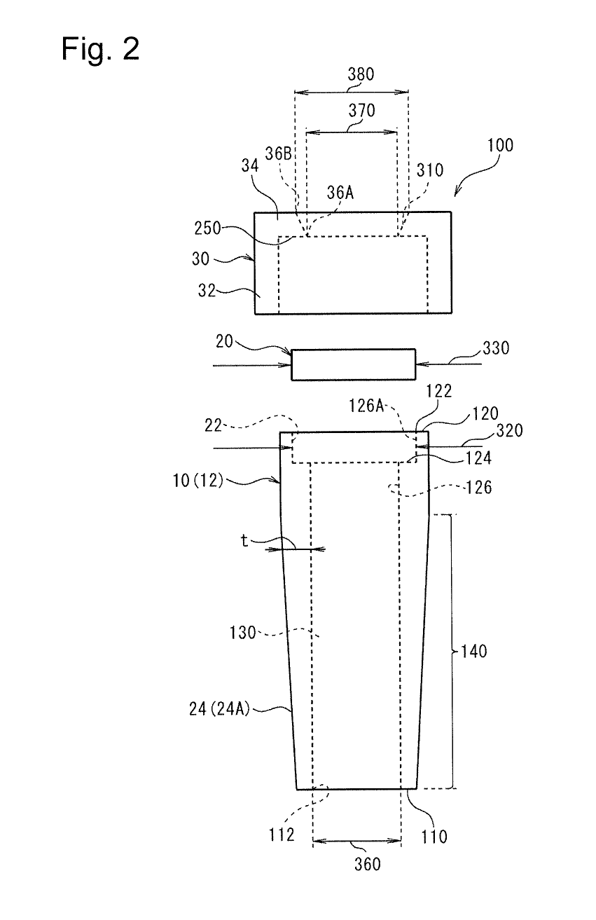

FIG. 2 is a side view of the culture vent plug 100 according to the first embodiment of the present invention, and illustrates a state where a vent plug body 20 and a lid body 30 are detached from a frame body 10.

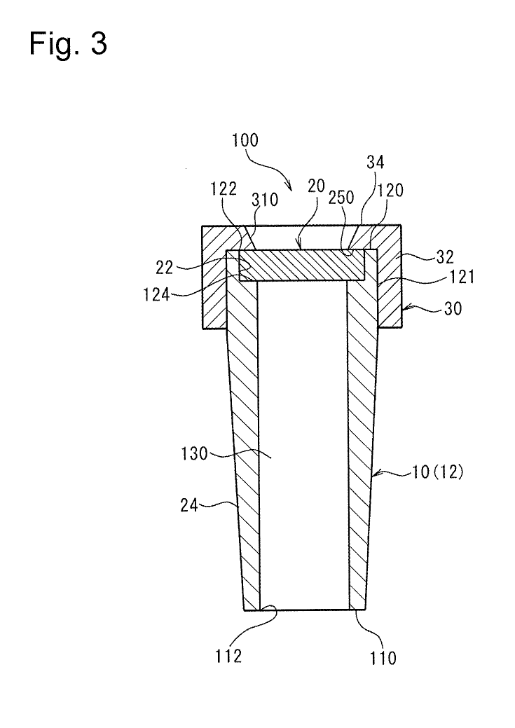

FIG. 3 is across-sectional view taken along line III-III of the culture vent plug 100 illustrated in FIG. 1. In FIG. 3, illustration of the culture vessel 900 is omitted.

As illustrated in FIG. 1, the culture vent plug 100 includes the vent plug body 20 and the frame body 10. The vent plug body 20 can secure ventilation between the inside and the outside of the culture vessel 900. The frame body 10 includes a holding portion 22 (see FIG. 3) holding the vent plug body 20, and an attachment portion 24 which can be attached to and detached from a vessel opening 910 of the culture vessel 900. The vent plug body 20 is held by the holding portion 22 so as to be capable of being repeatedly attached to and detached from the frame body 10.

In the present invention, the fact that the vent plug body 20 is capable of being repeatedly attached to and detached from the frame body 10 means as follows. That is, when the vent plug body 20 is detached from the frame body 10, mechanical destruction, chemical modification, physical deformation or the like does not occur in the frame body 10 and the vent plug body 20, and when the vent plug body 20 is held again by the frame body 10, the culture vent plug 100 in the state identical to the state before the vent plug body 20 is detached from the frame body 10 can be obtained.

For example, in the culture vent plug 100 according to the present embodiment, the entire interface between the holding portion 22 and the vent plug body 20 held by the holding portion 22 forms a microscopic air layer (illustration of which is omitted).

That is, the culture vent plug 100 does not have an adhesion layer or a fusion layer for integrating the frame body 10 and the vent plug body 20 in the above interface. Therefore, it is possible to detach the vent plug body 20 from the frame body 10 without destructing them, and it is possible to easily reproduce the state before the vent plug body 20 is detached from the frame body 10 when the vent plug body 20 is held again by the frame body 10.

More specifically, as illustrated in FIGS. 2 and 3, the culture vent plug 100 according to the present invention is configured such that the holding portion 22 is a recessed portion opened at an exposed end 120, which is one end of the frame body 10. Inner diameter 320 (see FIG. 2) of the above recessed portion is approximately uniform from the exposed end 120, which is the above one end of the frame body 10, to an inserted end 110, which is the other end of the frame body 10. Although illustration is omitted, inner diameter 320 of the above recessed portion may decrease as proceeding from the exposed end 120, which is the above one end, to the inserted end 110, which is the other end of the frame body 10.

The holding portion 22 can be appropriately designed such that the vent plug body 20 can be held in the frame body 10 without deviating from the scope and spirit of the present invention. For example, as illustrated in FIG. 2, the above recessed portion, which is the holding portion 22, is configured of a step 124 provided on an inner peripheral surface 126 of the frame body 10, and a base end portion inner peripheral surface 126A, which is part of the inner peripheral surface 126 and includes an exposed side opening 122.

As illustrated in FIG. 2, inner diameter 320 of the above recessed portion and diameter 330 of the vent plug body 20 are substantially identical, and therefore, the vent plug body 20 is contained and held in the above recessed portion. For example, the vent plug body 20 may be held in the holding portion 22 by friction force by bringing the peripheral surface of the above recessed portion into contact with the outer peripheral surface of the vent plug body 20. By fitting the vent plug body 20 into the recessed portion, the vent plug body 20 can be detachably attached to the frame body 10.

The above term, approximately identical, includes not only the case where inner diameter 320 of the above recessed portion and diameter 330 of the vent plug body 20 are completely identical, but also includes both the case where diameter 330 of the vent plug body 20 is slightly greater than inner diameter 320 of the recessed portion and the case where diameter 330 is slightly less than inner diameter 320.

For example, in the case where at least one of the frame body 10 and the vent plug body 20 is formed of an elastic member, it is possible to make diameter 330 of the vent plug body 20 greater than inner diameter 320 of the recessed portion. Thus, pressing force is generated between the base end portion inner peripheral surface 126A and the side surface of the vent plug body 20, and the state of holding the vent plug body 20 is stabilized.

In addition, although illustration is omitted, in the case of adopting an aspect of sandwiching the vent plug body 20 by the frame body 10 and the lid body 30 in the front-end to base-end direction using the lid body 30, which will be described later, or the like, the vent plug body 20 can be favorably held even when diameter 330 of the vent plug body 20 is less than inner diameter 320 of the recessed portion. In the case where diameter 330 of the vent plug body 20 is less than inner diameter 320 of the recessed portion, operation to detach the vent plug body 20 from the frame body 10, is easy.

Hereinafter, the vent plug body 20 will be described in detail. The vent plug body 20 can secure ventilation between the inside and outside of the culture vessel 900. The degree of air permeability of the vent plug body 20 is not particularly limited; however, for example, the above degree of air permeability is a degree of satisfying the aerobic property of an organisms such as microorganism or cell and the like to be cultured in the culture vessel 900.

### Organisms Such as Microorganism or Cell

For example, a ceramic may be used for the vent plug body 20.

The term, ceramic, herein means a porous ceramic exhibiting the above air permeability, which is pottery and porcelain in general including a metal oxide sintered body. The ceramic can be selected for example, from among zirconia, alumina, silicon carbide, and aluminum nitride; however, the ceramic is not limited to them. As the above ceramic which can be used for the present embodiment, the ceramic with porosity of, for example, 20% or higher and 80% or lower is preferable selected.

Ceramics generally have extremely high heat resistance of 1000° C. or higher, and in particular, 2000° C. or higher. Therefore, since the vent plug body 20, which can be detached from the frame body 10, is configured of a ceramic, it is possible to subject only the vent plug body 20 to a sterilization process at a high temperature of several hundred ° C., and furthermore 1000° C. or higher.

In the culture vent plug 100, during culture, dust and bacteria are likely to adhere to the vent plug body 20, which is an area through which a gas passes. Therefore, selecting a ceramic, which has high heat resistance, as the vent plug body 20 and enabling a sterilization process under a strict temperature condition is extremely advantageous from the viewpoint of aseptically performing culture.

From the viewpoint of subjecting the vent plug body 20 to a high-temperature sterilization process under a selectively strict condition in the culture vent plug 100, the heatproof temperature of a member configuring the vent plug body 20 is preferably higher than the heatproof temperature of the member configuring the frame body 10.

In the case where the heat resistance of the frame body 10 is lower than that of the vent plug body 20, the sterilization process of the frame body 10 may be performed separately from the sterilization process of the vent plug body 20. In addition, since the vent plug body 20 can be detached from the frame body 10 in the culture vent plug 100, in the case where the frame body 10 deteriorates before the vent plug body 20 which is made of a ceramic deteriorates, only the frame body 10 may be replaced with a new one, which is excellent in economic efficiency.

It is noted that the vent plug body 20 according to the present embodiment is not limited to be made of a ceramic but may be appropriately configured of a member having air permeability. A member which configures the vent plug body 20 other than a ceramic can be appropriately selected, for example, from among a rubber molding such as silicone rubber, a foamed resin molding such as a foamed chlorinated polyethylene molding and a foamed polyurethane re sin molding, and the like. The member configuring the vent plug body 20 has air permeability in any case. The above member having air permeability includes, for example, a continuous pore inside.

In the present embodiment, the vent plug body 20 can be configured to be harder than the frame body 10. The hardness of the vent plug body 20 and frame body 10 can be compared with each other by measuring Vickers hardness or durometer hardness of the vent plug body 20 and frame body 10 and comparing measured values of the vent plug body 20 and frame body 10 with each other. More specifically, in the case where both the vent plug body 20 and the frame body 10 are made of elastic bodies, it is possible to measure the hardness of them by means of a durometer and to compare hardness of them with each other. In addition, in the case where both the vent plug body 20 and the frame body 10 are made of hard members, the hardness of them may be measured by means of a Vickers hardness meter and hardness of them may be compared with each other. In the case where one of the vent plug body 20 and the frame body 10 is an elastic body and the other is a hard member such as a ceramic, it is preferable that the hardness be compared by using an identical hardness measuring instrument. It is preferred that the hardness be measured at fixed room temperature (for example, a temperature environment in the range from 20° C. or higher and 25° C. or lower).

In the present embodiment, by configuring the vent plug body 20 so as to be harder than the frame body 10, the following effect is exhibited. That is, when the culture vent plug 100 is attached to the vessel opening 910 of the culture vessel 900, the shape of the flexible frame body 10 follows the shape of the vessel opening 910, and therefore, the culture vent plug 100 can plug the vessel opening 910 with high airtightness. Since the vent plug body 20 is harder than the frame body 10, strain due to deformation of the frame body 10 whose shape has followed the shape of the vessel opening 910 is less likely to be transmitted to the vent plug body 20. Therefore, a vent hole formed inside the vent plug body 20 is hardly crushed and desired air permeability is secured.

The shape of the vent plug body 20 according to the present invention is a flat shape such as a disc shape, a pellet shape, and the like. Thickness of the flat-shaped vent Plug body 20 may be any appropriate thickness allowing ventilation between the inside and the outside of the culture vessel 900 and not allowing dust and bacteria to pass through the vent plug body 20, and is not particularly limited. It is needless to say that the shape of the vent plug body 20 may be any arbitrary shape other than the flat shape, such as a columnar body, a cube, a rectangular parallelepiped, or the like. Here, the term, thickness of the vent plug body 20, means the dimension of the vent plug body 20 in the front-end to base-end direction.

The diameter of the vent plug body 20 can be appropriately changed depending on the opening size of the symmetric culture vessel 900. For a vessel opening 910 with the outer diameter of about 18 mm, a disc-shaped vent plug body 20 made of a ceramic and having the diameter of about 14 mm and the thickness of about 6.5 mm can be prepared; however, this is only an example and does not limit the present invention in any way.

For example, as illustrated in FIG. 2, the vent plug body 20 according to the present embodiment is held by the frame body 10 such that the vent plug body 20 is positioned on the base end side with respect to a regulating portion 140. Therefore, the shape retention property of the area of the culture vent plug 100 on the base end side, exposed from the culture vessel 900 when the culture vent plug 100 is attached to the culture vessel 900 (hereinafter also referred to as a grip area) is excellent. Since such a grip area is provided, the grip area is gripped when the culture vent plug 100 is attached to or detached from the culture vessel 900 (see FIG. 1). Therefore, the culture vent plug 100 can be easily gripped and is excellent in operability. In particular, in the case where the vent plug body 20 is configured of a hard member such as a ceramic and the frame body 10 is configured of an elastic body such as silicone rubber, the above grip area is preferably provided. This is because when the grip area is gripped, the grip area can be securely gripped by because the frame body 10 is elastically deformed adequately, and the shape of the grip area is favorably retained because the vent plug body 20 serves as a core. Note that details of the regulating portion 140 will be described later.

Hereinafter, the frame body 10 will be described in detail. The frame body 10 according to the present embodiment is a member configuring the culture vent plug 100 together with the vent plug body 20, and constitutes by itself the frame body 10 for the culture vent plug.

That is, the frame body 10 is used for the culture vent plug 100 and includes the holding portion 22 repeatedly and detachably holding the vent plug body 20, and the attachment portion 24 detachably attached to the vessel opening 910 of the culture vessel 900.

In the culture vent plug 100, since the vent plug body 20 can be repeatedly attached to and detached from the frame body 10, it is possible to replace only the frame body 10 when the frame body 10 deteriorates. It is needless to say that in the case where the vent plug body 20 deteriorates before the frame body 10 deteriorates, only the vent plug body 20 may be replaced in the culture vent plug 100.

The attachment portion 24 is a predetermined length area including an inserted side opening 112 of the frame body 10 and extending in the front-end to base-end direction. The culture vent plug 100 according to the present embodiment is attached by being fitted into the vessel opening 910. As illustrated in FIG. 1, the outer peripheral surface of the frame body 10 is an attachment surface 24A, and a predetermined location of the attachment surface 24A directly or indirectly contacts the vessel opening 910, and therefore the frame body 10 is attached to the vessel opening 910.

In the culture vent plug 100 according to the present embodiment, the frame body 10 and the vent plug body 20 are provided at different areas in the front-end to base-end direction. Specifically, the holding portion 22 is provided on the exposed side opening 122 side with respect to the middle in the front-end to base-end direction, and the attachment portion 24 is provided on the inserted side opening 112 side. Due to such a configuration, when the culture vent plug 100 is attached to the culture vessel, the vent plug body 20 is hardly crushed and favorable air permeability is secured.

It is noted that although illustration is omitted, in a modification of the present embodiment, an attachment portion 24 including an area provided with a holding portion 22 may be provided. Specifically, an aspect where part or the whole of the holding portion 22 is fitted into a culture vessel 900 may be possible. In particular, in the case where a vent plug body 20 is formed of a hard member such as a ceramic, it is possible to secure sufficiently favorable air permeability even in the case where the holding portion 22 is fitted into the culture vessel 900.

The frame body 10 is a cylindrical body 12 including the inserted end 110 capable of being inserted into the vessel opening 910, the exposed end 120 exposed from the vessel opening 910, and a vent passage 130 interconnecting the inserted side opening 112 provided at the inserted end 110 and the exposed side opening 122 provided at the exposed end 120. As illustrated in FIG. 2, the frame body 10, which is the cylindrical body 12, includes the regulating portion 140 at an intermediate portion in the front-end to base-end direction, regulating the insertion depth of the frame body 10 into the culture vessel 900.

As illustrated in FIG. 2, the inner diameter of the exposed side opening 122 is configured to be greater than the inner diameter of the inserted side opening 112, and the vent plug body 20 is provided closer to the exposed side opening 122 than the regulating portion 140 is. Therefore, the vent plug body 20 is easily attached to and detached from the exposed side opening 122 side.

The above-described holding portion 22 is provided in an area of the vent passage 130 including the exposed side opening 122. The vent passage 130 is blocked by the vent plug body 20 held by the holding portion 22 in such a manner as to allow ventilation. The outer diameter at the inserted side opening 112 is configured to be less than the inner diameter of the vessel opening 910 of the culture vessel 900. Therefore, the attachment portion 24 of the frame body 10 is fitted into the culture vessel 900 (see FIG. 1).

As illustrated in FIG. 2, the regulating portion 140 according to the present embodiment is configured to have a tapered shape in which the outer diameter of the frame body 10 continuously increases as proceeding from the inserted end 110 to the exposed end 120. Wall thickness t of the regulating portion 140 configured to have the tapered shape continuously increases as proceeding from the inserted end 110 to the exposed end 120.

The outer diameter of the regulating portion 140 is tapered. In contrast, the inner diameter of the regulating portion 140 is approximately uniform in the front-end to base-end direction.

Thus, the portion in the vicinity of the inserted side opening 112 can be flexible so as to be easily inserted into the vessel opening 910, and the strength of the regulating portion 140 can be increased as proceeding from the portion to the exposed side opening 122. That is, the attached state of the above-described wall thickness t to the vessel opening 910 can be stabilized.

The tapered regulating portion 140 is a predetermined length area in the front-end to base-end direction. The regulating portion 140 can appropriately regulate the insertion depth of the frame body 10 into the culture vessel 900 in the vessel opening 910 of the culture vessel 900. The diameter dimension of the vessel opening 910 falls within the range greater than the outer diameter dimension of the regulating portion 140 on the front end side and less than or equal to the outer diameter dimension of the regulating portion 140 on the base end side.

Note that the culture vent plug 100 according to the present embodiment can appropriately adopt a configuration other than a tapered configuration for the tapered regulating portion 140. For example, although illustration is omitted, a protruding portion protruding toward the outside in the radial direction is provided on the outer peripheral surface of the frame body 10, and the insertion depth of the frame body 10 into the culture vessel 900 may be regulated by bringing the protruding portion into contact with the vessel opening 910.

The member configuring the frame body 10 is not particularly limited; however, for example, the frame body 10 according to the present embodiment includes an elastic body. Here, the fact that the frame body 10 includes an elastic body includes the case where the entirety of the frame body 10 is configured of an elastic body and the case where an elastic body is provided on part of the frame body 10.

Examples of the elastic body include a rubber elastic body, a foam resin body, and the like.

Examples of the rubber elastic body include synthetic rubber containing a resin material, such as silicone rubber, ethylene propylene diene rubber, urethane rubber, nitrile rubber, and fluoro rubber, natural rubber, and the like; however, the rubber elastic body is not limited to the above mentioned rubber. In particular, silicone rubber is preferable because the moldability is favorable and can easily obtain desirable elasticity as the frame body 10. Here, the term, silicone rubber means a rubber member made of a synthetic resin mainly containing silicone (silicon resin).

Rubber hardness of the above rubber elastic body is measured by means of a durometer. More specifically, for example, the rubber hardness can be measured by means of a type A durometer in compliance with JIS K 6253. The rubber hardness of the above rubber elastic body is not particularly limited; however, for example, the rubber hardness preferably falls within the range from 40 A or more and 80 A or less.

Examples of the foam resin body include a foamed resin molding such as a silicone foam body, a foamed chlorinated polyethylene molding, a foamed polyurethane resin molding, and the like; however, the foam resin body is not limited to them.

Hardness of the above foam resin body can be measured by a durometer. For example, a durometer suitable for measuring sponge hardness can be selected. Durometer hardness of the foam resin body is not particularly limited; however, for example, the durometer hardness is preferably falls within the range from 20 or more and 45 or less. Hardness of the foam resin body may be measured in compliance with, for example, Japan Rubber Association Standard SRIS 0101.

By using a rubber molding such as silicone rubber as the frame body 10, it is possible to enhance the close-contact property of the frame body 10 with respect to the vessel opening 910 and to improve airtightness between the frame body 10 and the culture vessel opening.

As described, the constituent member of the frame body 10 can also be determined by taking into consideration hardness and heat resistance of the constituent member of the vent plug body 20.

The dimensions of the frame body 10 can be appropriately determined by taking into consideration the inner diameter or the outer diameter of the vessel opening 910, the thickness and the diameter of the vent plug body 20, and the like.

For example, the length of the regulating portion 140 in the front-end to base-end direction can be set to about 30 mm, the length from the base end side end portion of the regulating portion 140 to the exposed end 120 can be set to about 13 mm. The inner diameter of the frame body 10 in the regulating portion 140 may be set to about 11 mm, and the outer diameter may be appropriately determined according to the inner diameter of the vessel opening 910 of a target culture vessel 900. It is noted that the above dimensions are an example of the present embodiment, and do not limit the present invention in any way.

For example, in a plurality of frame bodies 10, holding portions 22 may adopt common dimensions fitted to a prescribed vent plug body 20, and the outer diameter dimension of the attachment portion 24 of one frame body 10 may differ from that of another frame body 10. Thus, by using the common vent plug body 20 and replacing a frame body 10 with a frame body 10 different in the dimension of the attachment portion 24, it is possible to apply the culture vent plug 100 to various culture vessels 900 different from each other in the inner diameter of the vessel opening 910. An aspect of replacing a frame body 10 with a frame body 10 different in the dimension of the attachment portion 24 while using the common vent plug body 20, is an advantage which can be achieved in the present embodiment in which the vent plug body 20 can be repeatedly attached to and detached from the frame body 10.

A plurality of frame bodies 10 with identical dimensions of the holding portions 22 and different dimensions of the attachment portions 24 can also be handled as a kit.

Note that in a culture vent plug 100 of a type to be externally fitted to a vessel opening 910, which will be described later as a second embodiment, in a plurality of frame bodies 10, the same effect as described above can be obtained by preparing the plurality of frame bodies 10 in which the dimensions of holding portions 22 are identical and inner diameter dimensions of attachment portions 24 are different from each other.

Next, the lid body 30 will be described in detail.

As illustrated in FIGS. 1 to 3, the culture vent plug 100 further includes the lid body 30. The lid body 30 is repeatedly and detachably attached to an exposed end portion 121 of the frame body 10 which includes the exposed end 120, and from which the vent plug body 20 is exposed (see FIG. 3). The lid body 30 includes a wall portion 32 provided in the circumferential direction, and a ceiling portion 34 extending inward in the radial direction from one end portion of the wall portion 32. The ceiling portion 34 includes a ceiling open hole 310 from which the vent plug body 20 is exposed.

The exposed end portion 121 is an area which is part of the frame body 10, includes the exposed end 120, and extends by a predetermined length in the front-end to base-end direction.

The above-described term, one end portion of the wall portion 32 means a predetermined length area including one end surface of the wall portion 32. The ceiling portion 34 includes both an aspect of extending inward in the radial direction from the one end surface of the wall portion 32, or an aspect of extending inward in the radial direction from the vicinity of the one end surface of the wall portion 32.

As illustrated in FIG. 2, the lid body 30 includes the wall portion 32 provided in the circumferential direction, and the ceiling portion 34 extending inward in the radial direction from the one end portion of the wall portion 32. The ceiling portion 34 includes the ceiling open hole 310 from which the vent plug body 20 is exposed. The side of the lid body 30 facing the ceiling open hole 310 is opened.

The opening diameter of the ceiling open hole 310 in the present embodiment is configured to be approximately identical to the inner diameter of the inserted side opening 112. Therefore, the lid body 30 does not limit the quantity of airflow allowed by the frame body 10 fitted into the vessel opening 910.

As illustrated in FIG. 2, the ceiling open hole 310 in the present embodiment is a hole having a predetermined length in the front-end to base-end direction, and the opening diameter of the ceiling open hole 310 increases as proceeding from the front end to the base end. Opening diameter 370 of a lower end opening 36A is less than opening diameter 380 of an upper end opening 36B. Opening diameter 370 of the lower end opening 36A in the present embodiment is the minimal opening diameter of the ceiling open hole 310 and may be approximately identical to opening diameter 360 of the inserted side opening 112.

That is, in the present embodiment, as illustrated in FIG. 2, the diameter of the ceiling open hole 310 of the lid body 30 decreases as proceeding from the upper end side opening (upper end opening 36B) to the lower end side opening (lower end opening 36A).

In other words, the ceiling open hole 310 in the present embodiment is formed to be tapered from the lower end opening 36A toward the upper end opening 36B, and opening diameter 380 of the upper end opening 36B is greater than opening diameter 370 of the lower end opening 36A.

Such an aspect exhibits the following effect in comparison with the case where the opening diameter of the ceiling open hole 310 is approximately identical from the upper end opening 36B to the lower end opening 36A. That is, in the above aspect, a surface 250 of the ceiling portion 34 on the vent plug body 20 side can be made larger, and opening diameter 380 of the upper end opening 36B can be designed to be large. Therefore, the property of holding the vent plug body 20 sandwiched by the frame body 10 and the lid body 30 can be increased without limiting the quantity of airflow passing through the vent plug body 20. In addition, according to such an aspect, gas or water vapor exhausted from inside the culture vessel 900 to which the culture vent plug 100 is attached hardly retains on the periphery of the lower end opening 36A, and the gas or the water vapor is easily released to the outside along the tapered shape of the ceiling open hole 310.

For example, the culture vent plug 100 may also be configured such that the opening diameter of the ceiling open hole 310 of the lid body 30 is greater than opening diameter 360 of the inserted side opening 112 of the frame body 10. Therefore, in comparison with the aspect where opening diameter 360 of the inserted side opening 112 of the frame body 10 is approximately identical to opening diameter 380 of the upper end opening 36B of the lid body 30, ventilation between the inside of the culture vessel 900 to which the culture vent plug 100 is attached (see FIG. 1) and outside air can be more smoothly performed. In an aspect where the opening diameter of the ceiling open hole 310 is not uniform, the maximal opening diameter of the ceiling open hole 310 is preferably greater than the opening diameter of the inserted side opening 112 of the frame body 10.

In addition, for example, the culture vent plug 100 may be configured such that the opening diameter of the ceiling open hole 310 of the lid body 30 is less than opening diameter 360 of the inserted side opening 112 of the frame body 10. Therefore, ventilation between the inside of the culture vessel 900 to which the culture vent plug 100 is attached and outside air is secured, and the likelihood of dust or bacteria in the outside air passing through the vent plug body 20 and being mixed into the inside of the culture vessel 900 can be reduced. In an aspect where the opening diameter of the ceiling open hole 310 is not uniform, the maximal opening diameter of the ceiling open hole 310 is less than the opening diameter of the inserted side opening 112 of the frame body 10.

In the culture vent plug 100 according to the present embodiment, the ceiling open hole 310, the vent plug body 20, and the vent passage 130 are continuous so as to allow ventilation (see FIG. 3).

The inner peripheral surface of the wall portion 32 is brought into contact with the outer peripheral surface of the holding portion 22, and therefore, the lid body 30 is externally fitted to the frame body 10. The ceiling portion 34 covers the surface of the vent plug body 20 on the base end side. Opening diameter 370 of the lower end opening 36A is configured to be less than diameter 330 of the vent plug body 20. Therefore, the lid body 30 prevents the vent plug body 20 from being dropped off from the holding portion 22.

As illustrated in FIG. 3, the inner peripheral surface of the frame body 10, which is the cylindrical body 12, includes the step 124 formed by discontinuously increasing the inner diameter as proceeding from the inserted end 110 to the exposed end 120. The culture vent plug 100 according to the present embodiment sandwiches the vent plug body 20 by means of the step 124 and the lid body 30. Therefore, the attached state of the vent plug body 20 in the culture vent plug 100 is stabilized. More specifically, the vent plug body 20 is placed on the step 124 and is sandwiched by the step 124 and the inner side surface of the ceiling portion 34 of the lid body 30.

In the present embodiment, the outer diameter of the vent plug body 20 is approximately equal to the inner diameter of the exposed side opening 122. Thus, in the state where the lid body 30 is detached, the vent plug body 20 can be easily attached to and detached from the frame body 10. For example, a user of the culture vent plug 100 can easily disassemble the culture vent plug 100 into the frame body 10, the vent plug body 20, and the lid body 30 and clean them, perform a high-temperature sterilization process as necessary, and then, aseptically assemble the culture vent plug 100 in a clean bench.

The member configuring the lid body 30 is not particularly limited. For example, the lid body 30 can be configured by using a member similar to the member configuring the frame body 10. It is noted that in the culture vent plug 100, the constituent members of the lid body 30 and the frame body 10 may be identical or different from each other. In addition, the lid body 30 may be formed of a resin suitable for injection molding, such as a vinyl chloride resin, a polyethylene terephthalate resin, and the like.

In a preferred example of the present embodiment, for example, each of the frame body 10 and the lid body 30 is configured of a rubber elastic body, and rubber hardness of the frame body 10 is less than rubber hardness of the lid body 30. Therefore, it is possible to provide the culture vent plug 100 in which the close-contact property of the frame body 10 with respect to the culture vessel 900 is favorable and the shape retention property of the outer circumferential shape of the portion exposed from the culture vessel 900 is high.

The rubber elastic body configuring the frame body 10 and the rubber elastic body configuring the lid body 30 may be of the same kind or different kinds. Here, the term, rubber elastic bodies of the same kind, means rubber elastic bodies in which main configurations of resins constituting the rubber elastic bodies are identical. For example, rubber polymer compounds mainly containing silicone are of the same type even though they differ from each other in rubber hardness. In contrast, a rubber polymer compound mainly containing silicone and a rubber polymer compound having a urethane bond structure in a molecule are of different types.

The above rubber elastic body is similar to the rubber elastic body which has been described above in relation to the frame body 10, and therefore detailed description of the rubber elastic body will be omitted here.

When rubber hardness of the rubber elastic body configuring the frame body 10 is compared with rubber hardness of the rubber elastic body configuring the lid body 30, the method for measuring rubber hardness is not particularly limited. Rubber hardness of the rubber elastic body configuring the frame body 10 and rubber hardness of the rubber elastic body configuring the lid body 30 may be measured in any way as long as they are measured under the same condition. Examples of the method for measuring rubber hardness include measurement using a type A durometer in compliance with JIS K 6253. In the present embodiment, in the case where rubber hardness is measured by means of the type A durometer, rubber hardness of the rubber elastic body configuring the frame body 10 is preferably greater than or equal to 40 A and less than 60 A, and rubber hardness of rubber elastic body configuring the lid body 30 is preferably greater than or equal to 60 A and less than or equal to 80 A.

Incidentally, culture experiments are usually performed under a plurality of culture conditions such as using a plurality of culture vessels and making culture conditions differ from each other. At that time, it is possible to visually and easily discriminate culture vessels under different culture conditions by color-coding a plurality of lid bodies 30 and using the same lid color for identical culture conditions.

The present invention includes a lid body kit (illustration of which is omitted) including a plurality of lid bodies 30. For example, in the plurality of lid bodies 30 included in the lid body kit, the color of the outer peripheral surface of one lid body 30 differs from the color of the outer peripheral surface of another lid body 30. The lid body 30 included in the lid body kit is used for the culture vent plug 100 and can be repeatedly and detachably attached to the exposed end portion 121 of the frame body 10, exposed from the vessel opening 910. The lid body 30 used for the lid kit widely includes an aspect without the function of sandwiching the vent plug body 20 or the aspect of fitting to an arbitrary location on the outer periphery of the frame body 10, exposed from the vessel opening 910.

More specifically, for example, in the case where culture experiments are performed in three experimental systems in which three levels of culture medium concentration are set using test tubes, a lid body 30 whose outer peripheral surface is red can be used for a test tube used for an experimental section at a first culture medium concentration, a lid body 30 whose outer peripheral surface is green can be used for a test tube used for an experimental section at a second culture medium concentration, and a lid body 30 whose outer peripheral surface is yellow can be used for a test tube used for an experimental section at a third culture medium concentration. Therefore, it is possible to distinguish at a glance test tubes under different culture conditions from one another without marking each test tube with a sticker or the like.

### Second Embodiment

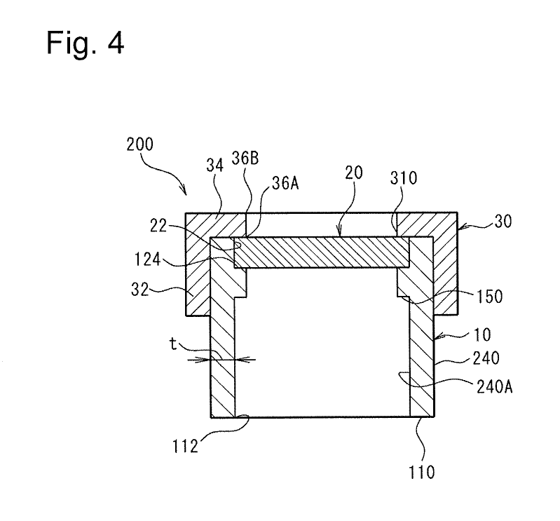

Hereinafter, a culture vent plug 200, which is a second embodiment of the culture vent plug according to the present invention, will be described with reference to FIG. 4.

FIG. 4 is a cross-sectional view of the culture vent plug 200 according to the second embodiment. The cutting direction of the cross section is identical to line illustrated in FIG. 1. The present embodiment differs from the first embodiment in the point to be described below. The configuration of the first embodiment can be appropriately adopted for the rest of the configuration of the present embodiment.

In the culture vent plug 200, the inner diameter of an inserted side opening 112 is approximately equal to an outer diameter of a vessel opening 910 (see FIG. 1). The culture vent plug 200 includes a holding portion 22 and an attachment portion 240. The holding portion 22 is located on the base end side with respect to the attachment portion 240. The inner peripheral surface of the frame body 10 in the attachment portion 240 is an attachment surface 240A. A culture vessel 900 (see FIG. 1) is inserted into the inserted side opening 112 so that the attachment surface 240A is brought into contact with the vessel opening 910 of the culture vessel 900 and the outer peripheral surface of the culture vessel 900 in the vicinity of the vessel opening 910. The culture vent plug 200 can be externally fitted to the culture vessel 900.

A regulating portion 150, which is a step whose diameter discontinuously decreases as proceeding from the front end to the base end, is provided on the inner peripheral surface of the culture vessel 900. The inner diameter of the culture vent plug 200 is approximately identical from the inserted side opening 112 to the regulating portion 150. The vessel opening 910 of the culture vessel 900 inserted inside the culture vent plug 200 in the direction from the inserted side opening 112 toward the base end is brought into contact with the regulating portion 150, and therefore insertion depth of the culture vessel 900 into the culture vent plug 200 is regulated.

The culture vent plug 200 is provided with a step 124 whose diameter discontinuously increases in the direction from the front end toward the base end is provided on the base end side with respect to the regulating portion 150. The holding portion 22 is configured of the step 124 and the inner peripheral surface of the frame body 10 positioned on the base end side with respect to the step 124. The vent plug body 20 is held by the holding portion 22.

As illustrated in FIG. 4, a ceiling open hole 310 according to the present embodiment is a hole with a predetermined length in the front-end to base-end direction, and the diameter of the ceiling open hole 310 is identical from the front end to the base end. That is, the opening diameter of a lower end opening 36A and the opening diameter of an upper end opening 36B in the culture vent plug 200 are approximately identical.

By making the dimensions of the holding portions 22 identical in the culture vent plug 100 according to the first embodiment and the culture vent plug 200 according to the second embodiment, the culture vent plug 100 and the culture vent plug 200 can be replaced with each other and used for the common vent plug body 20.

The first embodiment and the second embodiment according to the present invention have been described above. The configuration of one embodiment can be appropriately used for another embodiment. In addition, the above description does not limit the present invention in any way, and the present invention allows the above embodiments to be appropriately changed and to include an additional configuration without deviating from the scope and spirit of the present invention.

### Third Embodiment

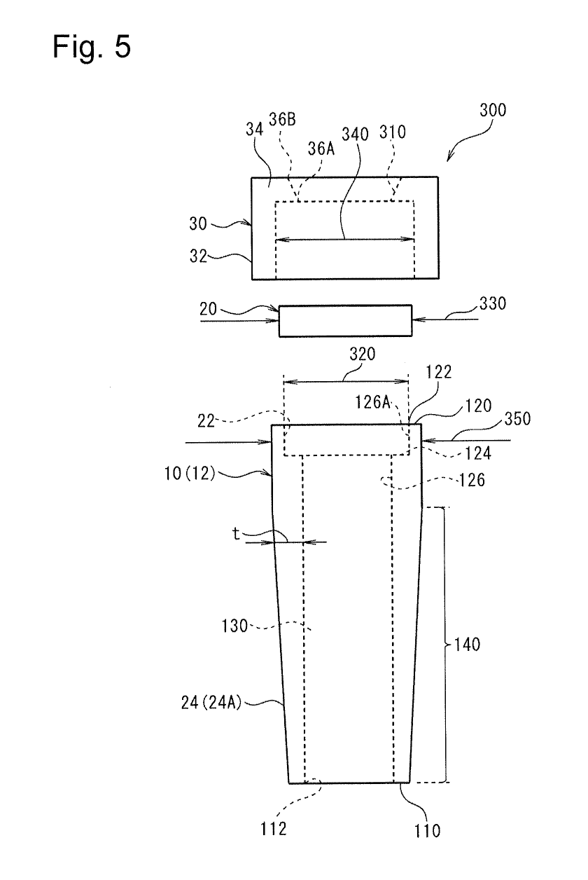

Hereinafter, a culture vent plug 300, which is a third embodiment of the culture vent plug according to the present invention, will be described with reference to FIG. 5.

FIG. 5 is a side view illustrating the culture vent plug 300 according to the third embodiment, and illustrates a state where a vent plug body 20 and a lid body 30 are detached from a frame body 10. The present embodiment differs from the first embodiment in the point to be described below. The configuration of the first embodiment can be appropriately adopted for the rest of the configuration of the present embodiment.

The culture vent plug 300 includes the frame body 10 configured of a rubber elastic body, the lid body 30 configured of a rubber elastic body, and the vent plug body 20 configured of a ceramic.

The lid body 30 covers an exposed end portion 121 (see FIG. 3). The vent plug body 20 is press-fitted into a holding portion 22, and the exposed end portion 121 is press-fitted into the lid body 30.

The term, press-fit, means to push in by applying pressure. The vent plug body 20 press-fitted into the holding portion 22 is held by the holding portion 22 in a state of being pressed by the holding portion 22. In addition, the exposed end portion 121 and the lid body 30 press-fitted into the exposed end portion 121 push against each other due to their pressing forces and are closely fitted to each other.

More specifically, the culture vent plug 300 is configured such that diameter 330 of the vent plug body 20 is greater than inner diameter 320 of the holding portion 22, which is a recessed portion opened at one end of the frame body 10, in a state where the vent plug body 20 and the lid body 30 are detached from the frame body 10. In addition, the culture vent plug 300 is configured such that outer diameter 350 of an exposed end 120 is greater than or equal to inner diameter 340 of the lid body 30.

Therefore, the vent plug body 20 attached to the frame body 10 is held in a state where the holding portion 22 formed of a rubber elastic body is expanded outward in the radial direction. Consequently, outer diameter 350 of the exposed end 120 of the frame body 10 extends outward in the radial direction. The above recessed portion of the lid body 30 is fitted to outer diameter 350 extended outward in the radial direction, and therefore inner diameter 340 of the lid body 30 is extended outward in the radial direction. As a result, the holding portion 22 and the vent plug body 20 are brought into closer contact with each other and the vent plug body 20 is securely held. In addition, the lid body 30 and the frame body 10 are brought into closer contact with each other. Therefore, the lid body 30 can be prevented from easily dropping off from the frame body 10.

In the present embodiment, the difference between diameter 330 of the vent plug body 20 and inner diameter 320 of the holding portion 22, which is a recessed portion opened at one end of the frame body 10, is not particularly limited; however, for example, the difference is preferably 0.5 mm or greater and 3 mm or less. Since diameter 330 of the vent plug body 20 is greater than inner diameter 320 of the holding portion 22 by 0.5 mm or greater, the held state of the vent plug body 20 when the vent plug body 20 is attached to the holding portion 22 can be significantly improved. In addition, by setting the value obtained by subtracting inner diameter 320 of the holding portion 22 from diameter 330 of the vent plug body 20 to 3 mm or less, deterioration of the holding portion 22 in the case of repeatedly attaching and detaching the vent plug body 20 to and from the frame body 10 can be suppressed.

### Fourth Embodiment

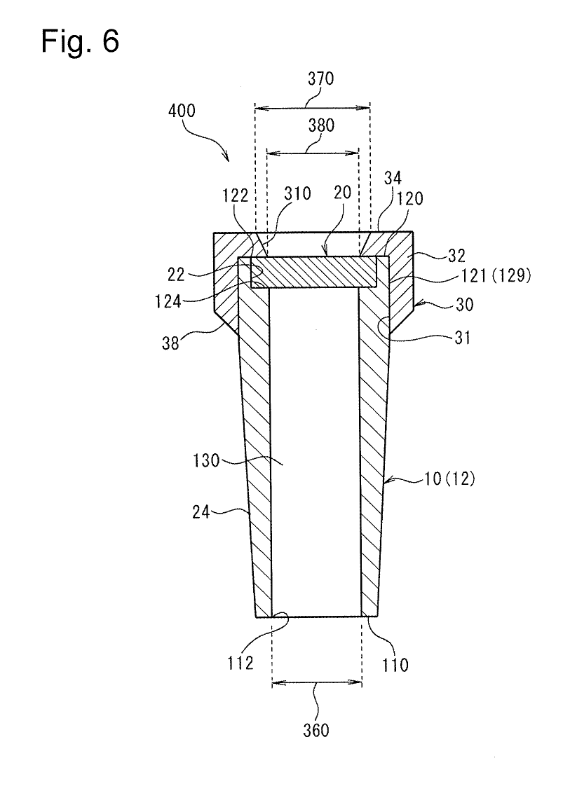

Hereinafter, a culture vent plug 400, which is a fourth embodiment of the culture vent plug of the present invention, will be described with reference to FIG. 6.

FIG. 6 is a cross-sectional view of the culture vent plug 400 exemplifying the culture vent plug according to the fourth embodiment. The cutting direction of the cross section is identical to line illustrated in FIG. 1. The present embodiment differs from the first embodiment in the point to be described below. The configuration of the first embodiment can be appropriately adopted for the rest of the configuration of the present embodiment.

In the culture vent plug 400 of the present embodiment, an outer peripheral surface 129 of an exposed end portion 121 of a frame body 10 and an inner peripheral surface 31 of a lid body 30 are both flat. In addition, in the culture vent plug 400, the thickness of a lower end portion 38 of a wall portion 32 of the lid body 30 becomes continuously or discontinuously thinner as proceeding from up to down.

In other words, the outer diameter of the lid body 30 decreases as proceeding from up to down in the lower end portion 38 of the wall portion 32, and the lower end portion 38 has a tapered shape tapered downward.

The above term, flat, does not require smoothness when a surface is microscopically observed, but means being flat to such a degree that there is no intentionally provided unevenness. The lower end portion 38 illustrated in FIG. 6 illustrates an example where the lower end portion 38 becomes continuously thinner as proceeding from up to down.

In the culture vent plug 400 having such a configuration, the lid body 30 can be easily attached to and detached from the frame body 10. More specifically, it does not happen that the outer peripheral surface 129 of the exposed end portion 121 of the frame body 10 and the inner peripheral surface 31 of the lid body 30 in the present aspect are hooked to each other due to physical unevenness or the like. In addition, since the lower end portion 38 of the wall portion 32 of the lid body 30 becomes thinner as proceeding from up to down, the lower end portion 38 is more flexible than the other area of the wall portion 32. Therefore, the lid body 30 is easily fitted to and detached from the exposed end portion 121.

In addition, since the lower end portion 38 of the wall portion 32 is thin, the culture vent plug 400 also has the following effect. That is, in the culture vent plug 400 attached to a culture vessel 900 (see FIG. 1), when a grip area exposed from the culture vessel 900 is gripped, a finger is less likely to be hooked on the lower end portion 38 of the wall portion 32, and not only the lid body 30 but also the entirety of the culture vent plug 400 is easily gripped. Therefore, operability when the culture vent plug 400 is attached to or detached from the culture vessel 900 is favorable.

### Advantageous Usage Example of Invention

In the culture vent plug according to the present invention, the vent plug body can be repeatedly attached to and detached from the frame body. Due to such a feature, the culture vent plug according to the present invention exhibits various advantageous effects that a conventional culture vent plug does not have. Note that the following description illustrates an example of the advantageous usage example of the present invention and does not limit the present invention in any way.

First, a user can simplify the structure by disassembling the culture vent plug according to the present invention used for a culture vessel into the vent plug body and the frame body. By washing the vent plug body and the frame body separately, the user can realize that the culture vent plug according to the present invention is more easily and more cleanly washed than a conventional culture vent plug in which a vent plug body and a frame body are integrally configured.

Second, the user can subject the vent plug body and the frame body which have been separately cleaned to a high-temperature sterilization process in a state where they are separated from each other. At that time, taking into consideration heat resistance of each of the vent plug body and the frame body, the vent plug body and the frame body may be subjected to the high-temperature sterilization process at temperatures different from each other, respectively. For example, since the vent plug body is a portion through which a gas flows into and out of the culture vessel, dust and bacteria easily adhere to it. Therefore, the vent plug body is desirably sterilized especially sufficiently in the culture vent plug. Therefore, when the culture vent body is configured of a member high in heat resistance (for example, a ceramic), the vent plug body can be subjected to the high-temperature sterilization process at a selectively high temperature. In contrast, in consideration of heat resistance or the like of the member configuring the frame body, the condition for the high-temperature sterilization process for the frame body may be appropriately set.

Third, after the high-temperature sterilization process, the user can dry the vent plug body and the frame body which have been separately subjected to the high-temperature sterilization process, in a state where the vent plug body and the frame body are separated from each other. The user can sufficiently dry the vent plug body and the frame body in a state where they are separated from each other in a shorter time than in the case of a conventional culture vent plug.

Fourth, the vent plug body and the frame body which have been sufficiently dried as described above are easily reassembled into the culture vent plug by the user, and are used again as the culture vent plug.

Fifth, after the user uses the culture vent plug according to the present invention for the culture vessel and cleans and dries the culture vent plug repeatedly a plurality of times, the user can replace one of the frame body and the vent plug body which has deteriorated earlier with a new one. Therefore, the culture vent plug according to the present invention is excellent also in terms of economic efficiency. As a preferred example, the vent plug body may be configured of a ceramic and the frame body may be configured of an elastic member such as rubber and a soft resin. Thus, the vent plug body can be subjected to a high-temperature sterilization process at a sufficiently high temperature, and the culture vent plug can be attached to the culture vessel with high airtightness due to flexibility of the frame body. In addition, in the case where the vent plug body made of a ceramic is attached to the frame body made of an elastic body such as rubber, the vent plug body is protected from physical shock by the frame body.

The above embodiments include the following technical ideas.

(1) A culture vent plug including:

a vent plug body capable of securing ventilation between inside and outside a culture vessel; and

a frame body including a holding portion holding the vent plug body, and an attachment portion capable of being attached to and detached from a vessel opening of the culture vessel, wherein

the vent plug body is held by the holding portion so as to be capable of being repeatedly attached to and detached from the frame body.

(2) The culture vent plug according to (1), wherein an entire interface between the holding portion and the vent plug body held by the holding portion is a microscopic air layer.

(3) The culture vent plug according to (1) or (2), wherein

the holding portion is a recessed portion opened at one end of the frame body, and

an inner diameter of the recessed portion is approximately uniform or decreases as proceeding from the one end of the frame body to another end of the frame body.

(4) The culture vent plug according to any one of (1) to (3), wherein hardness of the vent plug body is greater than hardness of the frame body.

(5) The culture vent plug according to any one of (1) to (4), wherein the vent plug body is made of a ceramic.

(6) The culture vent plug according to any one of (1) to (5), wherein the frame body includes an elastic body.

(7) The culture vent plug according to any one of (1) to (6), wherein the frame body is a cylindrical body including:

an inserted end capable of being inserted into the vessel opening;

an exposed end exposed from the vessel opening;

a vent passage interconnecting an inserted side opening provided at the inserted end and an exposed side opening provided at the exposed end; and

a regulating portion provided in an intermediate portion in a front-end to base-end direction and regulating insertion depth of the frame body into the culture vessel.

(8) The culture vent plug according to (7), wherein

an inner diameter of the exposed side opening is greater than an inner diameter of the inserted side opening, and

the vent plug body is provided closer to the exposed side opening than the regulating portion is.

(9) The culture vent plug according to (7) or (8), wherein

the regulating portion is configured to have a tapered shape such that an outer diameter of the frame body continuously increases as proceeding from the inserted end to the exposed end, and

a wall thickness of the regulating portion configured to have the tapered shape continuously increases as proceeding from the inserted end to the exposed end.

(10) The culture vent plug according to any one of (7) to (9) further including:

a lid body repeatedly and detachably attached to an exposed end portion of the frame body which includes the exposed end, and from which the vent plug body is exposed, the lid body including

a wall portion provided in a peripheral direction, and

a ceiling portion extending from one end portion of the wall portion inward in a radial direction, the ceiling portion including a ceiling open hole from which the vent plug body is exposed.

(11) The culture vent plug according to (10), wherein

an inner peripheral surface of the frame body, which is the cylindrical body, is provided with a step formed by discontinuously increasing an inner diameter of the step as proceeding from the inserted end to the exposed end, and

the vent plug body is sandwiched between the step and the lid body.

(12) The culture vent plug according to (10) or (11), wherein an opening diameter of the ceiling open hole is approximately identical to an inner diameter of the inserted side opening of the frame body.

(13) The culture vent plug according to any one of (10) to (12), wherein a diameter of the ceiling open hole of the lid body decreases as proceeding from an opening on an upper end side of the ceiling open hole to an opening on a lower end side of the ceiling open hole.

(14) The culture vent plug according to any one of (10) to (13), wherein

each of the frame body and the lid body is configured of a rubber elastic body, and

rubber hardness of the frame body is less than rubber hardness of the lid body.

(15) The culture vent plug according to any one of (10) to (14) including:

the frame body configured of a rubber elastic body;

the lid body configured of a rubber elastic body; and

the vent plug body configured of a ceramic; wherein

the lid body covers the exposed end portion, and

the vent plug body is press-fitted into the holding portion and the exposed end portion is press-fitted into the lid body.

(16) The culture vent plug according to any one of (10) to (15), wherein an opening diameter of the ceiling open hole of the lid body is greater than an opening diameter of the inserted side opening of the frame body.

(17) The culture vent plug according to any one of (10) to (15), wherein an opening diameter of the ceiling open hole of the lid body is less than an opening diameter of the inserted side opening of the frame body.

(18) The culture vent plug according to any one of (10) to (17), wherein an outer peripheral surface of the exposed end portion of the frame body and an inner peripheral surface of the lid body are both flat, and

thickness of a lower end portion of the wall portion of the lid body continuously or discontinuously decreases as proceeding from up to down.

(19) A lid body kit including:

a plurality of lid bodies each of which is used for the culture vent plug according to any one of (1) to (18) and is repeatedly and detachably attached to an exposed end portion of the frame body exposed from the vessel opening, the lid bodies differing from each other in color of an outer peripheral surface.

(20) A frame body for a culture vent plug used for the culture vent plug according to any one of (1) to (18) including:

a holding portion repeatedly and detachably holding a vent plug body; and

an attachment portion detachably attached to a vessel opening of a culture vessel.

(21) The frame body according to (20) being a cylindrical body including:

an inserted end capable of being inserted into the vessel opening of the culture vessel;

an exposed end exposed from the vessel opening;

a vent passage interconnecting an inserted side opening provided at the inserted end and an exposed side opening provided at the exposed end; and

a regulating portion provided at an intermediate portion in a front-end to base-end direction and regulating insertion depth of the frame body into the culture vessel.

This application is based upon and claims the benefit of priority from Japanese Patent Application No. 2014-102574 filed on May 16, 2014, the entire contents of which are incorporated herein by reference.

1. A culture vent plug comprising:
a vent plug body capable of securing ventilation between inside and outside a culture vessel; and
a frame body including a holding portion holding the vent plug body, and an attachment portion capable of being attached to and detached from a vessel opening of the culture vessel, and an exposed end exposed from the vessel opening; and
a lid body repeatedly and detachably attached to an exposed end portion of the frame body which includes the exposed end, and from which the vent plug body is exposed, wherein
the vent plug body is held by the holding portion so as to be capable of being repeatedly attached to and detached from the frame body,
the lid body includes
a wall portion provided in a peripheral direction, and
a ceiling portion extending from one end portion of the wall portion inward in a radial direction, the ceiling portion including a ceiling open hole from which the vent plug body is exposed,
each of the frame body and the lid body is configured of a rubber elastic body and the vent plug body is configured of a ceramic, and
a diameter of the vent plug body is greater than an inner diameter of the holding portion, an outer diameter of the exposed end portion is greater than or equal to an inner diameter of the lid body, the vent plug body is press-fitted into the holding portion and the exposed end portion is press-fitted into the lid body, and thus the lid body covers the exposed end portion and a lower end of the lid body is located below a lower surface of the vent plug body in a state where the lid body covers the exposed end portion.

2. (canceled)

3. The culture vent plug according to claim 1, wherein
the holding portion is a recessed portion opened at one end of the frame body, and
an inner diameter of the recessed portion is decreases as proceeding from the one end of the frame body to another end of the frame body.

4. (canceled)

5. (canceled)

6. (canceled)

7. (canceled)

8. (canceled)

9. (canceled)

10. (canceled)

11. (canceled)

12. (canceled)

13. The culture vent plug according to claim 1, wherein a diameter of the ceiling open hole of the lid body decreases as proceeding from an opening on an upper end side of the ceiling open hole to an opening on a lower end side of the ceiling open hole.

14. The culture vent plug according to claim 1, wherein
rubber hardness of the frame body is less than rubber hardness of the lid body.

15. (canceled)

16. (canceled)

17. (canceled)

18. The culture vent plug according to claim 1, wherein an outer peripheral surface of the exposed end portion of the frame body and an inner peripheral surface of the lid body are both flat, and
thickness of a lower end portion of the wall portion of the lid body continuously or discontinuously decreases as proceeding from up to down.

19. (canceled)

20. (canceled)

21. (canceled)
